(19) United States
(12) Patent Application Publication
(10) Pub. No.: US 2026/0125642 A1
(43) Pub. Date: May 7, 2026

(54) CHICKEN SERUM-FREE MEDIUM FOR PROMOTING PROLIFERATION OF CHICKEN PRIMORDIAL GERM CELLS (PGCs) AND USE OF CHICKEN SERUM-FREE MEDIUM

(71) Applicant: YANGZHOU UNIVERSITY, Yangzhou (CN)

(72) Inventors: Qisheng ZUO, Yangzhou (CN); Xin LIU, Yangzhou (CN); Bichun LI, Yangzhou (CN); Wei GONG, Yangzhou (CN); Xiaoqian LV, Yangzhou (CN); Yingjie NIU, Yangzhou (CN); Kai JIN, Yangzhou (CN)

(73) Assignee: YANGZHOU UNIVERSITY, Yangzhou (CN)

(21) Appl. No.: 18/981,770

(22) Filed: Dec. 16, 2024

Related U.S. Application Data
(63) Continuation of application No. PCT/CN2024/130240, filed on Nov. 6, 2024.

(51) Int. Cl. C12N 5/0735

(52) U.S. Cl. CPC C12N 5/0611; C12N 2500/90; C12N 2501/998

(57) ABSTRACT

A chicken serum-free medium for promoting proliferation of chicken primordial germ cells (PGCs) and a use of the chicken serum-free medium are provided. In the chicken serum-free medium, ovotransferrin is used instead of chicken serum. The ovotransferrin is used instead of chicken serum in a PGC culture system, and ovotransferrin can keep the self-renewing of PGCs to provide a basis for exploring a stable culture system. The chicken serum-free medium can avoid the adverse effects caused by an unclear composition of chicken serum. In addition, the accurate use of various growth factors can provide excellent culture conditions and reduce the culture cost.

### CROSS REFERENCE TO THE RELATED APPLICATIONS

This application is a continuation application of International Application No. PCT/CN2024/130240, filed on Nov. 6, 2024, the entire contents of which are incorporated herein by reference.

### TECHNICAL FIELD

The present disclosure belongs to the field of biotechnologies, and specifically relates to a chicken serum-free medium for promoting proliferation of chicken primordial germ cells (PGCs) and a use of the chicken serum-free medium.

### BACKGROUND

In the field of genetic breeding, PGCs have become important cells for gene editing and genetic improvement due to their unique developmental potential and genetic characteristics. PGCs originate from the ectoderm, migrate to the genital ridge at the early stage of embryonic development, and differentiate into female or male gametes with the development of a sexual gland. These characteristics make PGCs a key cell type for modifying genotypes from the source to prepare gene-edited animals.

However, the in vitro culture and proliferation of PGCs has always been a technical challenge. The traditional culture methods are often faced with problems such as low cell proliferation efficiency and low cell survival rate, which seriously limit the application of PGCs in genetic breeding. Therefore, it is of great scientific significance and application value to explore novel culture systems and methods to improve the in vitro proliferation efficiency of PGCs.

### SUMMARY

Technical problem to be solved: In view of the above-mentioned technical problems, the present disclosure provides a chicken serum-free medium for promoting proliferation of chicken PGCs and a use of the chicken serum-free medium. Ovotransferrin is a main component of chicken serum. In addition, ovotransferrin is transferrin widely present in organisms, and can promote the growth and proliferation of cells. During the culture of cells, ovotransferrin can provide the iron required by the growth of cells, and is involved in a variety of cellular metabolic processes, thereby contributing to the proliferation and differentiation of cells. Under specific culture conditions, ovotransferrin can significantly promote the in vitro proliferation of many types of cells. When an appropriate amount of ovotransferrin is added to a PGC medium, PGCs are in the optimal state, the number of PGCs increases significantly, and there is no obvious cell debris, indicating a significant improvement effect for the proliferation efficiency of PGCs.

Technical solutions: In a first aspect, the present disclosure provides a chicken serum-free medium for promoting proliferation of chicken PGCs, where in the chicken serum-free medium, ovotransferrin is used instead of chicken serum.

Preferably, the chicken serum-free medium further includes a Dulbecco's Modified Eagle Medium (DMEM), ultrafiltration water, and additives; and the additives include a concentrated calcium chloride stock solution, a B-27™ additive, a GlutaMax additive, an MEM non-essential amino acids solution, 2-mercaptoethanol, an EmbryoMax® nucleoside, sodium pyruvate, albumin, sodium heparin, basic fibroblast growth factor, activin A, and a penicillin-streptomycin mixed solution.

Preferably, a concentration of the ovotransferrin in the chicken serum-free medium is 0.1 mg/mL to 1 mg/mL.

Further, the concentration of the ovotransferrin in the chicken serum-free medium is 0.5 mg/mL.

In a second aspect, the present disclosure provides a use of the chicken serum-free medium described in the first aspect in promoting proliferation of chicken PGCs.

In a third aspect, the present disclosure provides a method for promoting proliferation of chicken PGCs, including the following steps: inoculating the chicken PGCs into the chicken serum-free medium described in the first aspect, and culturing at 37° C. for a specified period of time until a required number of cells are produced.

Beneficial effects: The present disclosure discloses the use of ovotransferrin instead of chicken serum in a PGC culture system, and ovotransferrin can keep the self-renewing of PGCs to provide a basis for exploring a stable culture system. The chicken serum-free medium of the present disclosure can avoid the adverse effects caused by an unclear composition of chicken serum. In addition, the accurate use of various growth factors can provide excellent culture conditions and reduce the culture cost.

### BRIEF DESCRIPTION OF THE DRAWINGS

FIGS. 1A-1C show the morphological observation results of PGCs cultured without chicken serum and with chicken serum in the present disclosure, where FIGS. 1A-1B show the influence of chicken serum-containing culture and chicken serum-free culture on the morphology of chicken PGCs, and FIG. 1C is a statistical chart of numbers of cells after 3 d of culture;

FIGS. 2A-2F show the influence of ovotransferrin on the proliferation of PGCs in the present disclosure, where FIG. 2A shows the influence of estradiol and ovotransferrin at different concentrations on the morphology of PGCs, FIG. 2B is a statistical chart of numbers of cells cultured with estradiol for 3 d, FIG. 2C is a statistical chart of numbers of cells cultured with ovotransferrin at different concentrations for 3 d, FIG. 2D is a statistical chart of viabilities of PGCs cultured with ovotransferrin at different concentrations for 3 d that are detected by CCK-8, FIG. 2E is a statistical chart of proliferation results of cells that are detected by EdU, and FIG. 2F shows EdU detection results of PGCs cultured with ovotransferrin at different concentrations;

FIGS. 3A-3G show the influence of ovotransferrin on a cycle of PGCs in the present disclosure, where FIG. 3A shows proportions of PGCs in different phases of the cycle that are detected by flow cytometry, FIG. 3B shows statistics of proportions of PGCs in different phases of the cycle, FIG. 3C shows relative expression levels of cell cycle-associated genes in PGCs, FIG. 3D is a statistical chart of the influence of ovotransferrin at different concentrations on levels of CCND1 and CCNB proteins, FIG. 3E shows the influence of ovotransferrin at different concentrations on levels of CCND1 and CCNB proteins, FIG. 3F shows detection results of an anti-PCNA antibody in PGCs cultured with ovotransferrin at different concentrations, and FIG. 3G is a statistical chart of anti-proliferating cell nuclear antigen (PCNA) antibody-positive cells among cells cultured with ovotransferrin at different concentrations;

FIGS. 4A-4M show the influence of ovotransferrin on the adhesion and pluripotency of PGCs in the present disclosure, where FIG. 4A shows the influence of ovotransferrin at different concentrations on a relative expression level of a DAZL gene, FIG. 4B shows the influence of ovotransferrin at different concentrations on a relative expression level of an NANOG gene, FIG. 4C shows the influence of ovotransferrin at different concentrations on a relative expression level of a POUV gene, FIG. 4D shows the influence of ovotransferrin at different concentrations on a relative expression level of an SOX2 gene, FIG. 4E shows the influence of ovotransferrin at different concentrations on a relative expression level of a ZO-1 gene, FIG. 4F shows the influence of ovotransferrin at different concentrations on a relative expression level of an Occludin gene, FIG. 4G shows the influence of ovotransferrin at different concentrations on a relative expression level of a JAM-A gene, FIG. 4H shows the influence of ovotransferrin at different concentrations on a relative expression level of a Claudin-1 gene, FIG. 4I shows the influence of ovotransferrin at different concentrations on levels of ZO-1, Occludin, JAM-A, and Claudin-1 proteins, and FIGS. 4J-4M are statistical charts of influence of ovotransferrin at different concentrations on levels of ZO-1, Occludin, JAM-A, and Claudin-1 proteins;

FIGS. 5A-5G show the influence of ovotransferrin on the apoptosis of PGCs in the present disclosure, where FIG. 5A shows the influence of ovotransferrin at different concentrations on a relative expression level of a BAX gene, FIG. 5B shows the influence of ovotransferrin at different concentrations on a relative expression level of a BCL-2 gene, FIG. 5C shows the influence of ovotransferrin at different concentrations on a relative expression level of a Caspase3 gene, FIG. 5D shows the influence of ovotransferrin at different concentrations on a relative expression level of a Caspase9 gene, FIG. 5E shows the influence of ovotransferrin at different concentrations on a relative expression level of a C-myc gene, FIG. 5F shows the apoptosis of PGCs that is detected by flow cytometry, and FIG. 5G is a statistical chart of proportions of apoptotic cells in PGCs;

FIGS. 6A-6C show the influence of ovotransferrin on a PI3K-AKT-mTOR signaling pathway in PGCs in the present disclosure, where FIG. 6A shows the influence of ovotransferrin at different concentrations on relative expression levels of PI3K, AKT, and mTOR genes, FIG. 6B shows the influence of ovotransferrin at different concentrations on levels of PI3K, AKT, and mTOR proteins, and FIG. 6C shows statistical charts of influence of ovotransferrin at different concentrations on levels of PI3K, AKT, and mTOR proteins; and

FIGS. 7A-7H show the influence of ovotransferrin on the ferroptosis of PGCs in the present disclosure, where FIG. 7A shows the influence of ovotransferrin at different concentrations on a total level of glutathione (GSH), FIG. 7B shows the influence of ovotransferrin at different concentrations on a level of reduced GSH, FIG. 7C shows the influence of ovotransferrin at different concentrations on a level of reduced GSH, FIG. 7D shows the influence of ovotransferrin at different concentrations on a content of malondialdehyde (MDA), FIG. 7E shows the influence of ovotransferrin at different concentrations on an activity of superoxide dismutase (SOD), FIG. 7F shows the influence of ovotransferrin at different concentrations on a content of ferrous iron, FIG. 7G shows the influence of ovotransferrin at different concentrations on a relative expression level of an SLA7A11 gene, and FIG. 7H shows the influence of ovotransferrin at different concentrations on a relative expression level of a GPX4 gene.

### DETAILED DESCRIPTION OF THE EMBODIMENTS

The present disclosure will be described in detail below with reference to the accompanying drawings and specific examples.

### Example 1 Verification of the Influence of Chicken Serum on the Morphology of PGCs

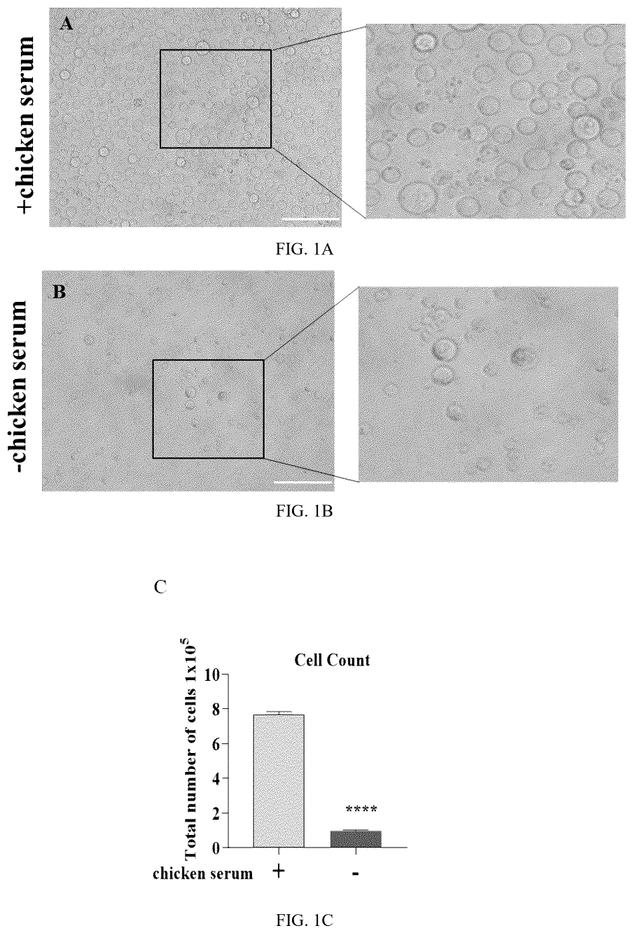

Primary cells were cultured for 70 d to successfully establish a cell line, and the cell line was frozen. The cell line was recovered before use. Specific components and amounts thereof in a medium were shown in Table 1 below. PGCs were cultured in the absence of chicken serum, and results were shown in FIG. 1A to FIG. 1B: PGCs can proliferate normally in the presence of chicken serum. However, PGCs do not proliferate in the absence of chicken serum, and the PGCs are flattened, broken, and irregular. These results indicate that chicken serum is indispensable in the culture of PGCs.

TABLE 1
Specific components of the medium for the 70 d culture

| Component | Concentration | | Volume | |
|---|---|---|---|---|
| DMEM | 75% | | 37.5 | mL |
| Ultrafiltration water | 24% | | 12 | mL |
| Concentrated calcium chloride stock | 0.15 | mM | 0.5 | mL |
| solution DMEM basal medium | | | 46.632 | mL |
| B-27 ™ additive | 1× | | 1 | mL |
| GlutaMax additive | 2 | mM | 0.5 | mL |
| MEM non-essential amino acids solution | 1× | | 0.5 | mL |
| 2-mercaptoethanol | 0.1 | mM | 91 | μL |
| Chicken serum | 0.2% | | 100 | μL |
| EmbryoMax ® nucleoside | 1× | | 0.5 | mL |
| Sodium pyruvate | 1.2 | mM | 0.6 | mL |

| | | | |
|---|---|---|---|
| Albumin | 0.2% | 0.1 | g |
| Sodium heparin | 0.01% | 50 | μL |
| Basic fibroblast growth factor | 1× | 20 | μL |
| Activin A | 1× | 25 | μL |
| Penicillin-streptomycin mixed solution | 1× | 0.5 | mL |

### Example 2 Promotion of Ovotransferrin on the Proliferation of PGCs in a Dose-Dependent Manner

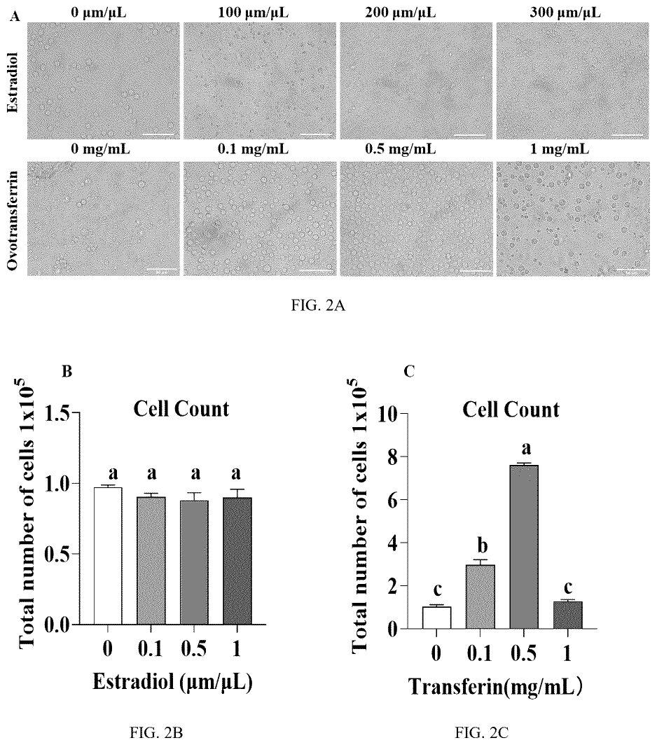

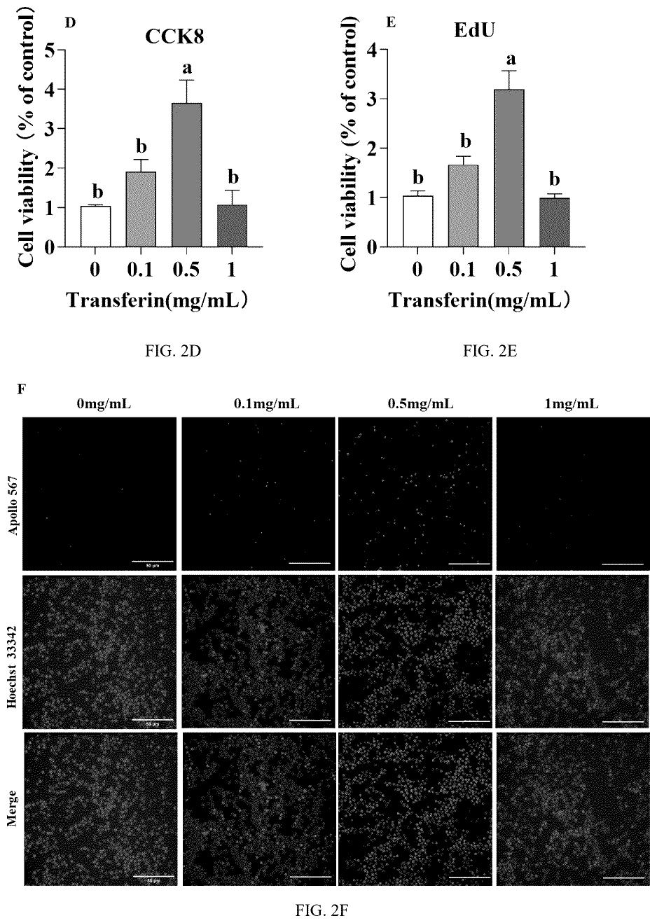

In order to determine the most suitable amount of ovotransferrin added during the culture of PGCs, estradiol and ovotransferrin were added at different concentrations to chicken serum-free media, PGCs were cultured with resulting media, and then the cell morphology was observed. Results were shown in FIG. 2A to FIG. 2C: Estradiol cannot promote the growth of PGCs. However, with the increase of a concentration of ovotransferrin, PGCs proliferate to varying degrees. With the increase of the concentration of ovotransferrin, the number of cells increases first and then decreases. When ovotransferrin is added at a medium concentration (0.5 mg/mL), PGCs exhibit a prominent state. There are significant cell debris and dead cells in PGCs exposed to a high concentration of ovotransferrin (1 mg/mL). However, compared with the addition of ovotransferrin at a concentration of 0.5 mg/mL, the addition of ovotransferrin at a low concentration (0.1 mg/mL) leads to a small PGC number, much cell debris, and a poor cell state.

According to the morphological observation of PGCs cultured with ovotransferrin at different concentrations: Ovotransferrin could promote the proliferation of PGCs. In order to further explore an impact of ovotransferrin on the proliferation of PGCs, a CCK-8 cell proliferation assay and an EdU cell proliferation assay were conducted. The CCK-8 cell proliferation assay was first conducted: PGCs were plated in a 24-well plate and passaged, and ovotransferrin was added at gradient concentrations (0 mg/mL, 0.1 mg/mL, 0.5 mg/mL, and 1 mg/mL). Cells were cultured in an incubator for 48 h, and then 100 μL of a resulting culture was pipetted from each well and added to a 96-well plate. 3 parallel wells were set for each group. 10 μL of a CCK-8 solution was added to each well (the generation of bubbles in each well should be avoided because the bubbles would affect the reading of an OD value). The 96-well plate was placed in a 37° C. and 5% CO₂ incubator and incubated for 2 h to 4 h. A wavelength of a microplate reader was adjusted to 450 nm. The 96-well plate was placed in the microplate reader, and the microplate reader was operated to read each absorbance value. Results were shown in FIG. 2D: With the increase of a concentration of ovotransferrin, a proliferation ability of PGCs increases first and then decreases. There is a significant difference between a medium-concentration ovotransferrin (0.5 mg/mL) group and other concentration groups (P<0.05), and there is no significant difference among the other concentration groups (P>0.05). Then the EdU chicken proliferation assay was conducted to detect the proliferation of PGCs: PGCs were plated in a 24-well plate and passaged, and ovotransferrin was added at gradient concentrations (0 mg/mL, 0.1 mg/mL, 0.5 mg/mL, and 1 mg/mL). An EdU solution was diluted with a PGC complete medium in a ratio of 1,000:1 to prepare an appropriate amount of a 50 μM EdU medium. 100 μL of the 50 μM EdU medium was added to each well, the plate was incubated for 2 h, and then the medium was discarded. Cells were washed 1 time to 2 times with phosphate buffered saline (PBS) for 5 min each time. Centrifugation was conducted at 1,400 rpm for 6 min, a resulting supernatant was removed with 20 μL of the supernatant left, and PGCs were pippetted up and down for thorough mixing and then dropped on a glass slide. 20 μL of a cell fixation solution (PBS including 4% of paraformaldehyde) was added dropwise on the glass slide, then the glass slide was incubated for 30 min at room temperature, and the fixation solution was discarded. 20 μL of a 2 mg/mL glycine solution was added dropwise on the glass slide, then the glass slide was incubated for 5 min, and then the glycine solution was discarded. 20 μL of PBS was added dropwise on the glass slide to allow washing for 5 min, and then the PBS was discarded. 20 μL of a permeating agent (PBS including 0.5% of TritonX-100) was added dropwise on the glass slide, and the glass slide was incubated for 10 min. Washing was conducted once with PBS for 5 min. 20 μL of a 1× Apollo staining reaction solution was added dropwise on the glass slide, the glass slide was incubated in the dark at room temperature for 30 min, and the staining reaction solution was discarded. 20 μL of a permeating agent was added dropwise to allow washing 2 times to 3 times for 10 min each time, and the permeating agent was discarded. Washing was conducted once with PBS for 5 min. 20 μL of a Hoechst 33342 staining solution was added dropwise on the glass slide, the glass slide was incubated in the dark at room temperature for 30 min, and then the staining solution was discarded. 20 μL of a permeating agent was added dropwise on the glass slide, and the glass slide was incubated for 10 min. Washing was conducted once with PBS for 5 min. Mounting was conducted with a neutral gum. The observation was conducted with different channels under a fluorescence microscope. Results were shown in FIG. 2E to FIG. 2F: With the increase of a concentration of ovotransferrin, a proliferation ability of PGCs increases first and then decreases. There is a significant difference between a medium-concentration ovotransferrin (0.5 mg/mL) group and other concentration groups (P<0.05), and there is no significant difference among the other concentration groups (P>0.05). It can be concluded that ovotransferrin can promote the proliferation of PGCs, and when ovotransferrin is added at a concentration of 0.5 mg/mL, PGCs exhibit the strongest proliferation ability.

### Example 3 Influence of Ovotransferrin on Genes and Proteins Related to a Cell Cycle, Apoptosis, a Signaling Pathway, and Cell Adhesion of PGCs

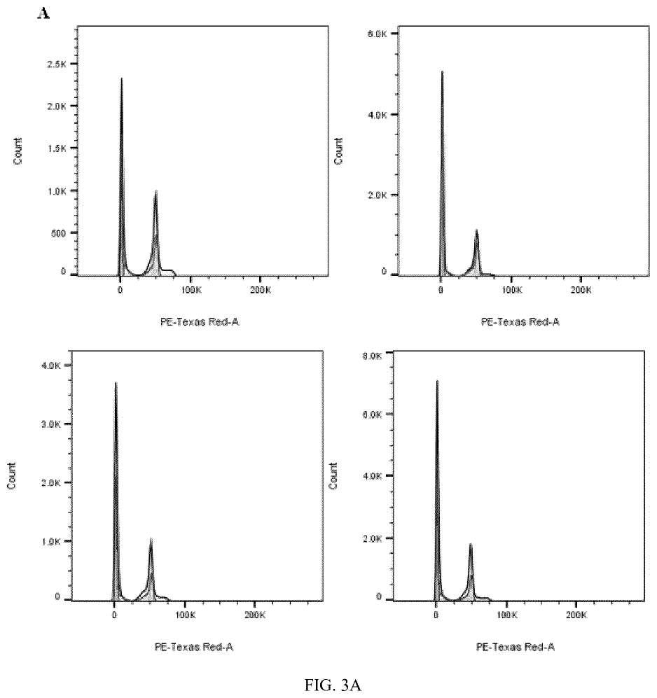

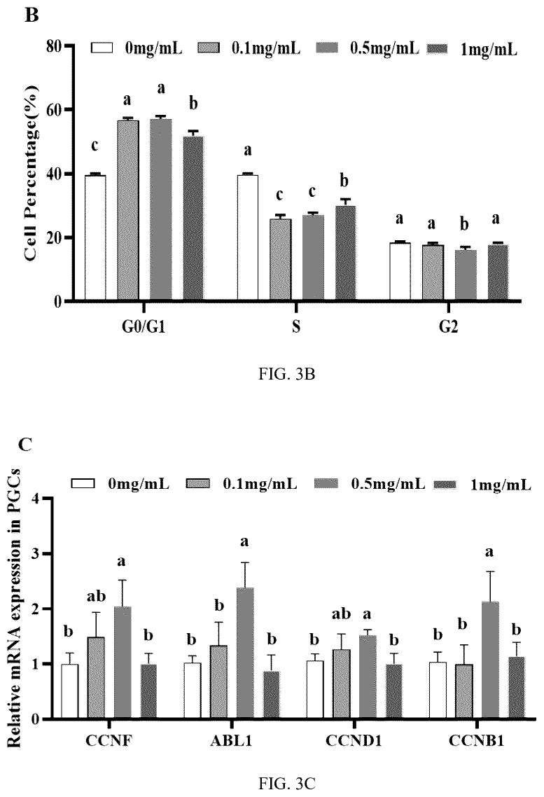

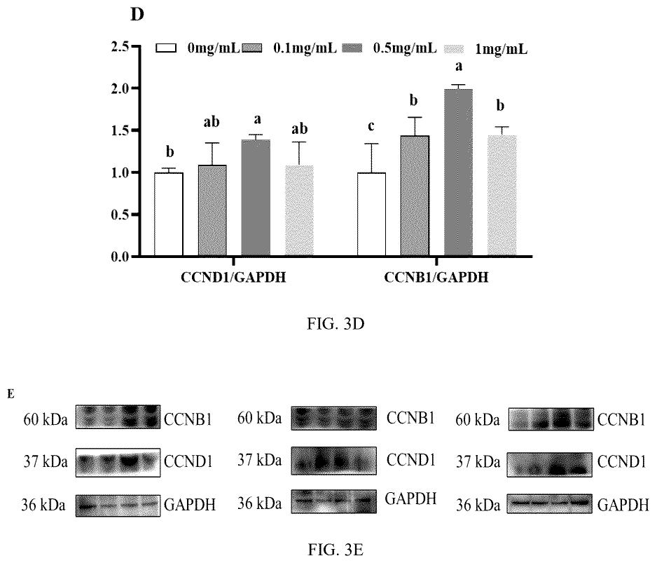

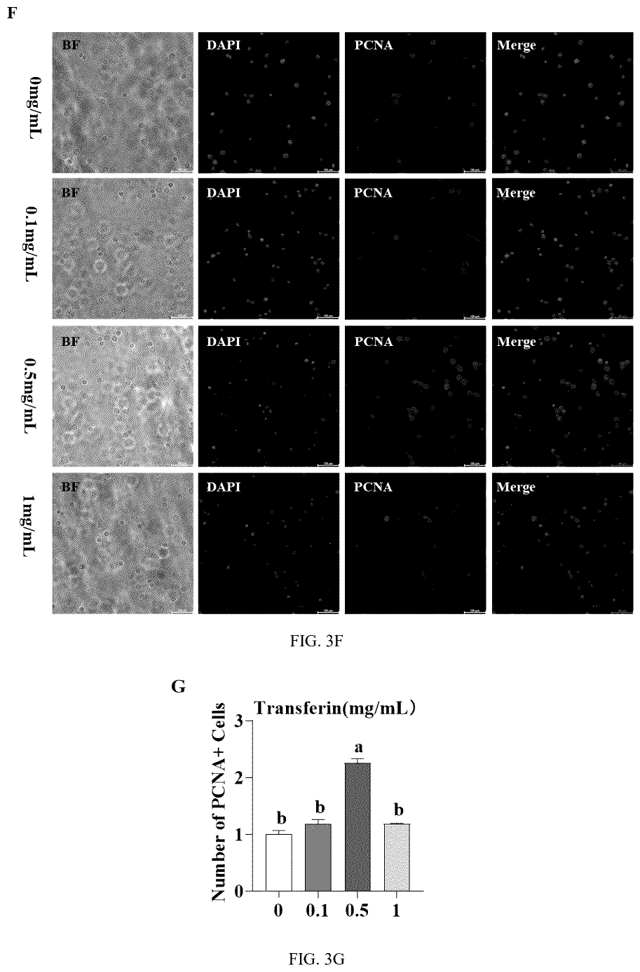

In order to further verify the influence of ovotransferrin on genes and proteins related to a cell cycle, apoptosis, a signaling pathway, and cell adhesion of PGCs, the expression of genes and proteins related to a cell cycle was detected by flow cytometry, quantitative reverse transcription polymerase chain reaction (qRT-PCR), and Western blot in this example, and results were shown in FIGS. 3A-3G to FIGS. 6A-6C.

When a cell cycle was detected by flow cytometry, cell fixation was conducted first: A cell pellet was thoroughly mixed with 1 mL of pre-cooled 70% ethanol gently to allow fixation at 4° C. for 2 h or more or overnight. Then the cells were settled through centrifugation at 1,000×g for 5 min, and then resuspended with 1 mL of pre-cooled PBS. Then the cells were settled through centrifugation at 1,000×g for 5 min. 10 μL of a propidium iodide stock solution and 10 μL of a RNase A solution were added to 0.5 mL of a staining buffer, and thorough mixing was conducted to prepare a propidium iodide staining solution for later use. 0.5 mL of the prepared propidium iodide staining solution was added to each cell sample, and thorough mixing was conducted gently to resuspend cells. The cells were incubated at 37° C. in the dark for 30 min and then tested at an excitation wavelength of 488 nm. The DNA content analysis and the light scattering analysis were conducted for the cells by flowjo analysis software. Results were shown in FIG. 3A to FIG. 3B: Compared with a chicken serum-free group (0 mg/mL), cells of the G0/G1 phase in the 0.5 mg/mL ovotransferrin group significantly increase, and cells of the S phase in the 0.5 mg/mL ovotransferrin group decrease, indicating that 0.5 mg/mL ovotransferrin can significantly promote the acceleration of a cell cycle and promote the cell proliferation.

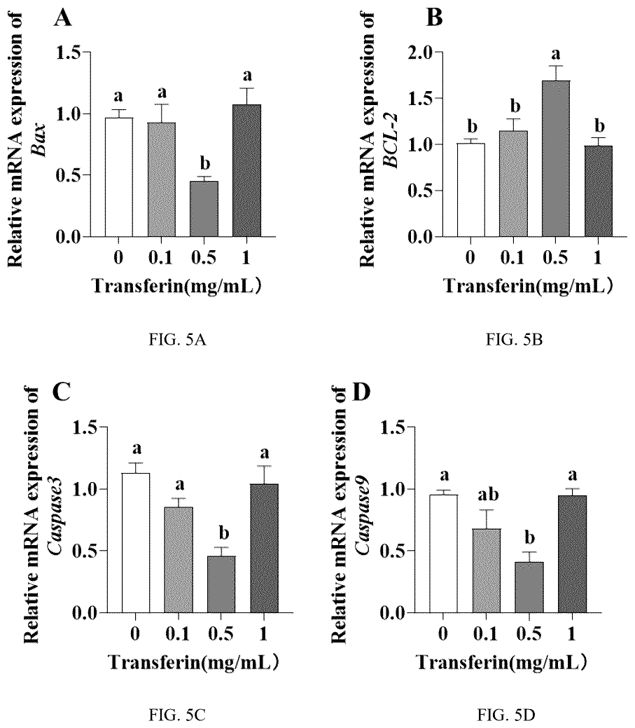

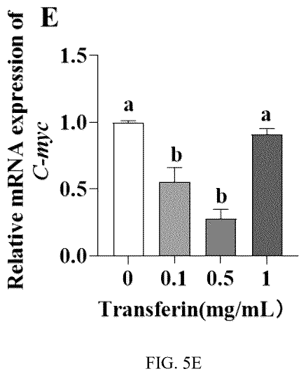

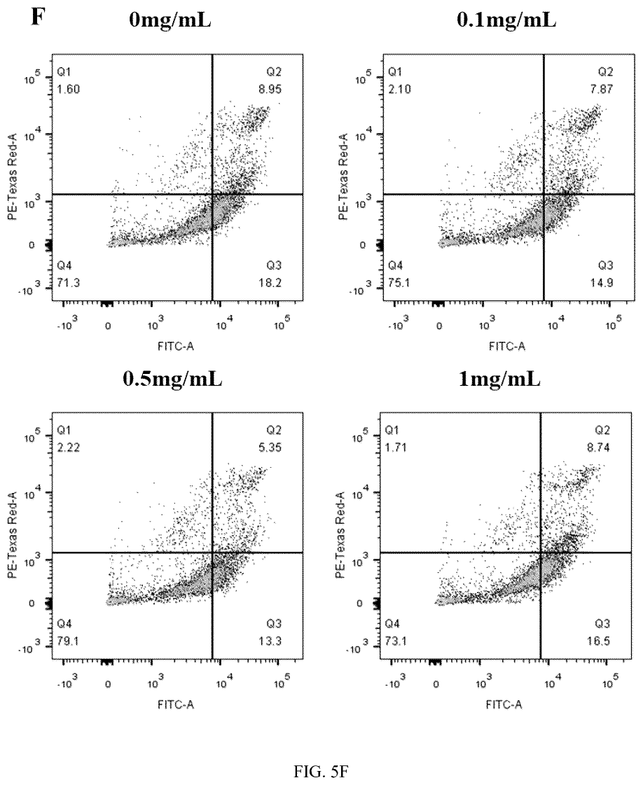

The apoptosis was detected by flow cytometry: PGCs were plated in a 24-well plate and passaged, ovotransferrin was added at gradient concentrations, and the plate was incubated for 72 h. Then PGCs were detected by flow cytometry. Specific steps were as follows: Cells were collected through centrifugation at 300×g and 4° C. The cells were washed twice with pre-cooled PBS, where centrifugation was conducted at 300×g and 4° C. for 5 min each time. 1×10⁵ to 5×10⁵ cells were collected. PBS was discarded, and 100 μL of 1× Binding Buffer was added to resuspend cells. 5 μL of Annexin V-FITC and 10 μL of a PI Staining Solution were added, and thorough mixing was conducted gently. A reaction was allowed in the dark at room temperature for 10 min to 15 min. 400 μL of 1× Binding Buffer was added, and thorough mixing was conducted to produce a test sample. The test sample was placed on ice, and then detected by flow cytometry within 1 h. Results were shown in FIGS. 5A-5G: With the increase of a concentration of ovotransferrin, an apoptosis rate of PGCs decrease first and then increase. An apoptosis rate of the 0.5 mg/mL ovotransferrin group significantly down-regulates compared with apoptosis rates of 0 mg/mL and 1 mg/mL ovotransferrin groups (P<0.05). There is no significant difference among other groups (P>0.05). The results show that ovotransferrin can inhibit the apoptosis of PGCs, and when ovotransferrin is added at a concentration of 0.5 mg/mL, there is the most significant inhibitory effect.

A PCNA proliferation assay was conducted through the following steps: A cell culture was collected in a 1.5 mL centrifuge tube and centrifuged at 1,400 rpm for 6 min, and a resulting supernatant was discarded. PBS was added for washing once, and then the PBS was discarded. 200 μL of 4% paraformaldehyde was added to allow fixation for 30 min. PBS was added for washing once, centrifugation was conducted at 1,400 rpm for 6 min, and a resulting supernatant was discarded. 200 μL of 0.5% TritonX-100 (diluted with PBS) was added to permeabilize cell membranes at room temperature for 20 min, and then PBS was added for washing once. Centrifugation was conducted at 1,400 rpm for 6 min, and a resulting supernatant was discarded. 10% FBS-PBS was slowly added to block at room temperature for 2 h. The blocking solution was removed, and TBST was added for washing once. 200 μL of diluted (1:200, using an antibody diluent) PCNA was added with a surface fully covered, and incubation was conducted overnight at 4° C. The next day, washing was conducted with TBST for 5 min. Then a fluorescent secondary antibody was added, and incubation was conducted in a 37° C. incubator for 2 h in the dark. The fluorescent secondary antibody was washed off with TBST. 200 μL of 4′,6-diamidino-2-phenylindole (DAPI) (5 ng/mL) was added to allow staining for 15 min, and then the DAPI was washed off. Mounting was conducted. Different fluorescence channels were observed under a fluorescence inverted microscope and photographed for recording, and results were shown in FIG. 3F to FIG. 3G. It can be known that 0.5 mg/mL ovotransferrin can significantly increase an anti-PCNA antibody-positive cell rate and promote the cell proliferation.

The following operations were conducted subsequently:

(1) Total RNA extraction: A PGC culture resulting from ovotransferrin culture was collected in a centrifuge tube and centrifuged at 1,400 rpm for 6 min, and a resulting supernatant was discarded. 1 mL of a TRIZOL reagent was added, and a resulting sample was pipetted up and down for thorough mixing, allowed to stand at 4° C. for 5 min, and then allowed to stand at room temperature for 5 min, such that proteins were fully dissociated. 0.2 mL of chloroform was added, a cap was tightened, and the centrifuge tube was vigorously shaken fully for 15 s, then allowed to stand at room temperature for 2 min to 3 min, and centrifuged at 12,000×g and 4° C. for 15 min such that layering occurred to produce an upper aqueous phase with RNA and a lower organic phase with proteins and DNA. 500 μL of the upper aqueous phase was taken, 0.5 mL of isopropanol was added, thorough mixing was conducted gently, and standing was allowed at room temperature for 10 min such that a gelatinous precipitate was produced at a bottom of a tube, which was RNA. Centrifugation was conducted at 4° C. and 12,000×g for 10 min, and a resulting supernatant was discarded. 1 mL of 75% ethanol was added to the precipitate, and thorough mixing was conducted gently. Centrifugation was conducted at 4° C. and 7500×g for 5 min, and a resulting supernatant was discarded. An RNA sample was air-dried, and an appropriate amount of enzyme-free water was added for dissolution (the dissolution could be promoted at 55° C. to 60° C. for 10 min). An RNA concentration was determined, and an OD₂₆₀/OD₂₈₀ ratio was detected and calculated by an ultraviolet spectrophotometer. An OD₂₆₀/OD₂₈₀ ratio of 1.9 to 2.0 indicated a prominent effect.

(2) cDNA synthesis: A template RNA was thawed on ice. 5×FastKing-RT SuperMix and RNase-Free ddH₂O were thawed at room temperature, and placed on ice immediately after thawing. Before use, each solution was vortexed for thorough mixing and transiently centrifuged to collect a liquid remaining on a wall of tube. A reverse transcription reaction system was prepared with the following components: 5×FastKing-RT SuperMix: 4 μL, Total RNA (depending on a concentration of RNA): 50 ng to 2 μg, and RNase-Free ddH₂O: making up to 20 μL. A reverse transcription reaction: a genome removal and reverse transcription reaction: 42° C., 15 min. An enzyme inactivation process: 95° C., 3 min. According to a content of RNA, RNase-Free ddH₂O was added to dilute cDNA.

(3) mRNA expression levels of each gene in PGCs treated with ovotransferrin at different concentrations were detected by qRT-PCR. A 10 μL qRT-PCR system included: 2× Universal SYBR Green Fast qPCR Mix: 5 μL, an upstream primer (10 μM): 0.4 μL, a downstream primer (10 μM): 0.4 μL, cDNA: 2 μL, and RNase-Free ddH₂O: making up to 10 μL. A reaction process: 95° C. for 3 min; and 95° C. for 5 s, and 60° C. for 30 s, with a total of 40 cycles. For PGC culture, three biological replicates were set, and three technical replicates were set for each sample. An expression level was quantified by a 2^{-ΔΔCT} method.

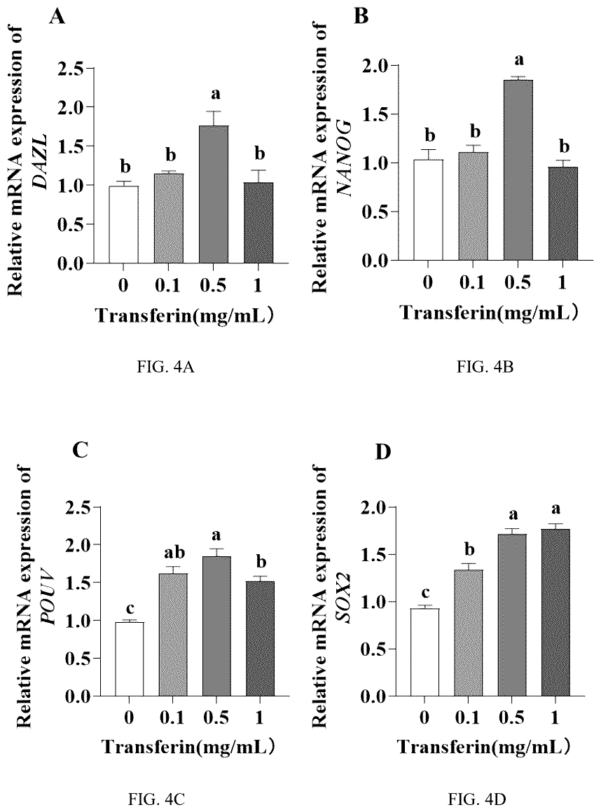

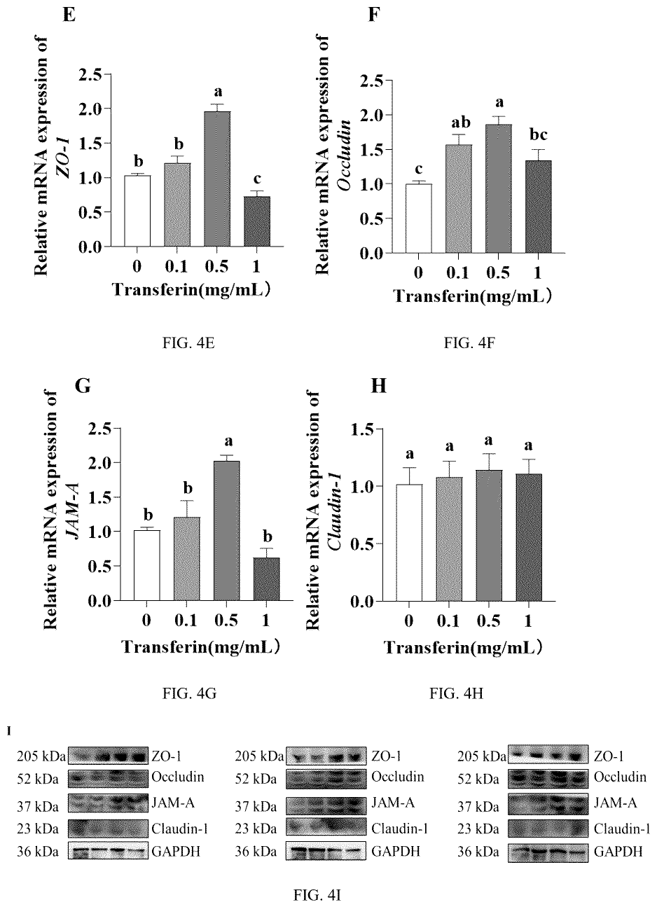

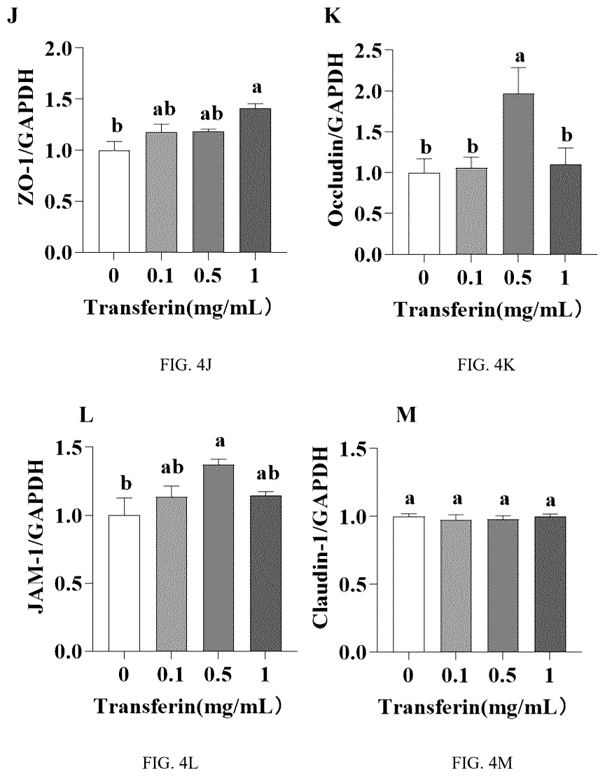

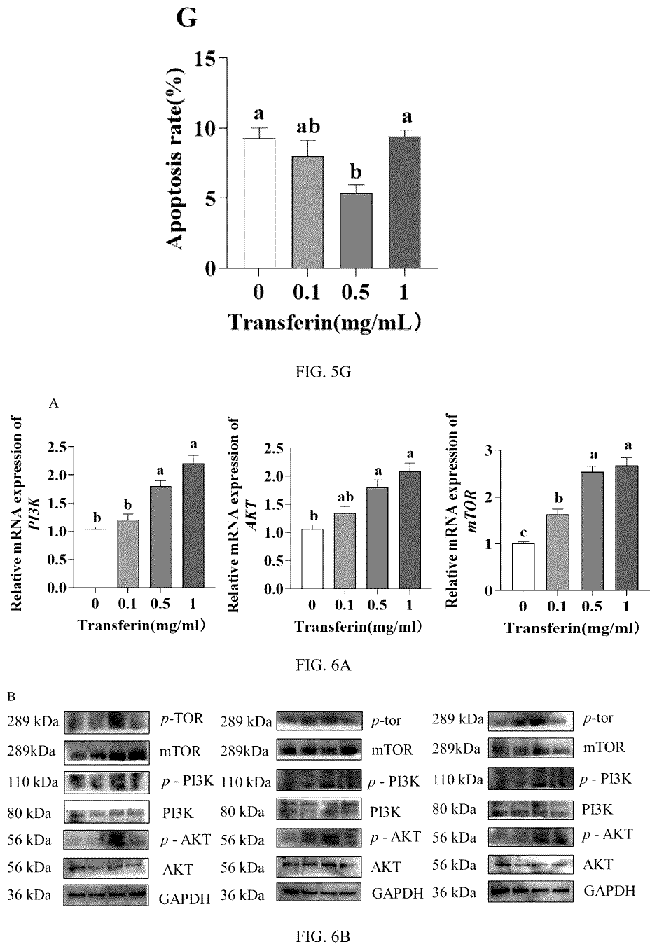

Western blot was then conducted: 1) Extraction of a total protein from PGCs: PGCs were plated in a 24-well plate and passaged, ovotransferrin was added at gradient concentrations, and the plate was incubated for 72 h. Centrifugation was conducted at 1,400 rpm for 6 min, and a resulting supernatant was discarded. 1 mL of a radioimmunoprecipitation assay (RIPA) buffer was added, and lysis was conducted for 40 min at a low temperature on ice. Then centrifugation was conducted for 10 min in a 4° C. centrifuge at 12,000 g, and a resulting supernatant was collected in a 1.5 mL centrifuge tube and stored at −80° C. 2) Determination of a protein concentration by a bicinchoninic acid (BCA) assay and preparation of a BCA working solution: According to a quantity of samples, 50 parts by volume of a BCA reagent A were thoroughly mixed with 1 part by volume of a BCA reagent B (50:1) to prepare an appropriate amount of a BCA working solution. The BCA working solution could be placed at room temperature for 24 h. Microwell assay of a protein concentration (20 μg/mL to 2,000 μg/mL): A 96-well plate was prepared. 25 μL of a BSA standard with each concentration was first added to each of 7 wells, and then 25 μL of a sample to be tested was added to a corresponding well according to a quantity of samples. 200 μL of a BCA working solution was added to each well, and thorough mixing was conducted, with 225 μL of a mixed solution per well in total. The plate was covered, incubated in a 37° C. incubator for 30 min to activate a reaction, and then cooled to room temperature. An absorbance value was detected at 562 nm by a microplate reader within 3 min to 5 min. 3) Preparation of WB reagents: Preparation of an electrophoresis solution: A pack of a sodium dodecyl sulfate (SDS) powder and 1,000 mL of ultrapure water were mixed and thoroughly stirred for 10 min. Preparation of a transfer solution: A pack of a transfer powder and 900 mL of ultrapure water were mixed and thoroughly stirred for 10 min, and then 100 mL of methanol was added. Preparation of a 1×TBST buffer: 500 mL of a 1×TBST buffer was prepared, and 25 mL of a 20×TBST buffer was thoroughly mixed with 475 mL of ultrapure water. 4) Sodium dodecyl sulfate-polyacrylamide gel electrophoresis (SDS-PAGE): The electrophoresis solution was pipetted with a pipette and used to gently blow loading holes of a gel plate. After the loading holes were dredged, loading was started. From left to right, a marker and protein samples to be tested were added successively. The loading time should be minimized to avoid the sample diffusion. An electrophoresis tank was turned on. A voltage of 80 V was set to conduct constant-voltage electrophoresis for about 40 min, and the voltage was adjusted to 110 V to conduct constant-voltage electrophoresis until running to a bottom of a separation gel. 5) Transfer: A polyvinylidene fluoride (PVDF) membrane was completely immersed in methanol for activation, and then transferred into the transfer solution and soaked for 10 min. Filter papers and black sponges were also soaked in the transfer solution. Gel cutting: A gel was cut off from a position corresponding to a size of a target protein, and front and back sides of the cut gel were remarked. The cut gel was placed in the transfer solution. A black sponge, 3 filter papers, the PVDF membrane, the gel, 3 filter papers, and a black sponge were arranged sequentially from bottom to top, and bubbles were squeezed out with a roller. During the arrangement in a transfer device, a black should correspond to a black transfer wall, a current should flow in a direction from a negative electrode to a positive electrode, and the current was 230 mA. A transfer time was determined according to a size of a protein. 6) Blocking: After the transfer was completed, a target protein with an appropriate size was cut, a blocking solution prepared just before use (50 mL of the blocking solution: 2.5 g of a skimmed milk powder+50 mL of 1×TBST), and thorough mixing was conducted. A resulting mixture was shaken for 10 min and incubated on a shaker for 2 h. After the blocking was completed, washing was conducted 3 times with 1×TBST for 10 min each time. 7) Incubation with a primary antibody: After the washing was completed, the primary antibody diluted with 1×TBST was added in a ratio of 1:1,000 to soak a PVDF membrane. A soaked PVDF membrane was incubated on a shaker for 10 min, and then placed at 4° C. overnight. The primary antibody was recovered the next day (which could be recycled 3 times) and washed 3 times with 1×TBST for 10 min each time. 8) Incubation with a secondary antibody: After the washing was completed, the secondary antibody diluted with 1×TBST was added in a ratio of 1:5,000 to soak a PVDF membrane. A soaked PVDF membrane was incubated on a shaker for 2 h. Then the secondary antibody was recovered (which could be recycled 3 times) and washed 3 times with 1×TBST for 10 min each time. 9) Electrogenerated chemiluminescence (ECL)-based color development: An ECL chromogenic solution was prepared with a solution A and a solution B in a ratio of 1:1. The ECL chromogenic solution was prepared just before use. About 100 μL of the chromogenic solution was added dropwise on each PVDF membrane evenly, and then the PVDF membrane was placed in a gel imaging system for color development and observed. A gray value was calculated with Image J software. Results were shown in FIG. 3D to FIG. 3E, FIG. 4A to FIG. 4I, and FIG. 6A to FIG. 6C: 0.5 mg/mL ovotransferrin can significantly improve the cell adhesion and the cyclin expression, activate a PI3K-AKT-mTOR signaling pathway, and promote the cell proliferation.

According to the above results: Ovotransferrin can promote the proliferation of PGCs. When ovotransferrin is added at a concentration of 0.5 mg/mL, PGCs exhibit the strongest proliferation ability. Ovotransferrin can also inhibit the apoptosis of PGCs, reduce the cell adhesion, and promote the activation of the PI3K-AKT-mTOR signaling pathway.

### Example 4 Influence of Ovotransferrin on the Ferroptosis of PGCs

Ferrous ion detection for PGCs: A reagent 1 was added to the collected cells with 0.2 mL of the reagent 1 for about 1×10⁶ cells, thorough mixing was conducted, and lysis was conducted on an ice box for 10 min. Centrifugation was conducted at 15,000×g for 10 min, and a resulting supernatant was collected for later use. Standard wells: 80 μL of each of standards with different concentrations was added to a corresponding well of a microplate plate. Assay wells: 80 μL of a sample to be tested was added to a corresponding well of the microplate plate. Control wells: 80 μL of a sample to be tested was added to a corresponding well of the microplate plate. 80 μL of a reagent 2 was added to each control well. 80 μL of a reagent 3 was added to each of the assay and standard wells. Thorough mixing was conducted, and the microplate plate was incubated at 37° C. for 10 min. An OD value of each well was determined at 593 nm by a microplate reader.

GSH and glutathione disulfide (GSSG) assays: Cells were collected through centrifugation at 1,400 rpm for 6 min and plated in a 96-well plate. A sample or a standard was added, and thorough mixing was conducted. 150 μL of a total GSH assay working solution was added, thorough mixing was conducted, and the plate was incubated at 25° C. or room temperature for 5 min. 50 μL of a 0.5 mg/mL nicotinamide adenine dinucleotide phosphate (NADPH) solution was added, and thorough mixing was conducted. The absorbance value at 405 nm was immediately determined with a microplate reader.

MDA assay: Through centrifugation at 1,400 rpm/min for 6 min, about 1×10⁶ cells were collected in a 1.5 mL centrifuge tube, 1 mL of a pre-cooled cell lysis buffer was added, and lysis was conducted on ice for 20 min during which the centrifuge tube was shaken vigorously for 30 s every 5 min. Centrifugation was conducted at 12,000×g for 5 min, and a resulting supernatant was collected. 1 mL of double-distilled water was added to the supernatant, and thorough mixing was conducted rapidly. A resulting mixture was boiled for 50 min in a boiling water bath (a small hole was formed in a cap of a tube before boiling to prevent the tube from bursting), quickly placed in ice water for cooling, and then centrifuged at 3,000 r/min and 4° C. for 15 min, and a resulting supernatant was collected and tested at 532 nm.

SOD assay: PGCs isolated and cultured were plated in a 24-well plate and passaged, ovotransferrin was added at gradient concentrations (0 mg/mL, 0.1 mg/mL, 0.5 mg/mL, and 1 mg/mL), and the plate was incubated for 72 h. Centrifugation was conducted at 1,400 rpm for 6 min, a resulting supernatant was discarded, and a resulting cell pellet was retained. 0.3 mL to 0.5 mL of a buffer (PBS or normal saline) was added to the cell pellet, and ultrasonic disruption was conducted with a power of 300 W in an ice water bath. During the ultrasonic disruption, an ultrasound was applied 4 times at an interval of 3 s to 5 s, and the ultrasound lasted for 30 s each time. Then an enzyme working solution and an enzyme dilution were added, and the assay was conducted with a microplate reader.

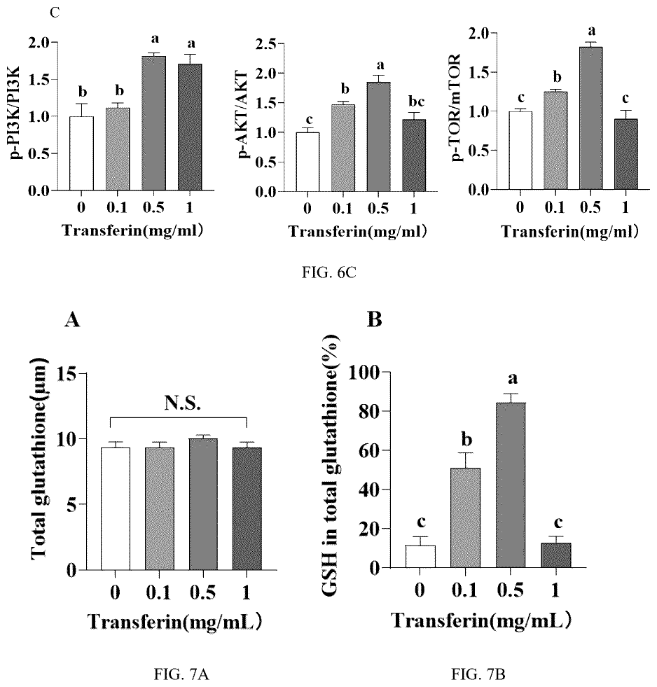

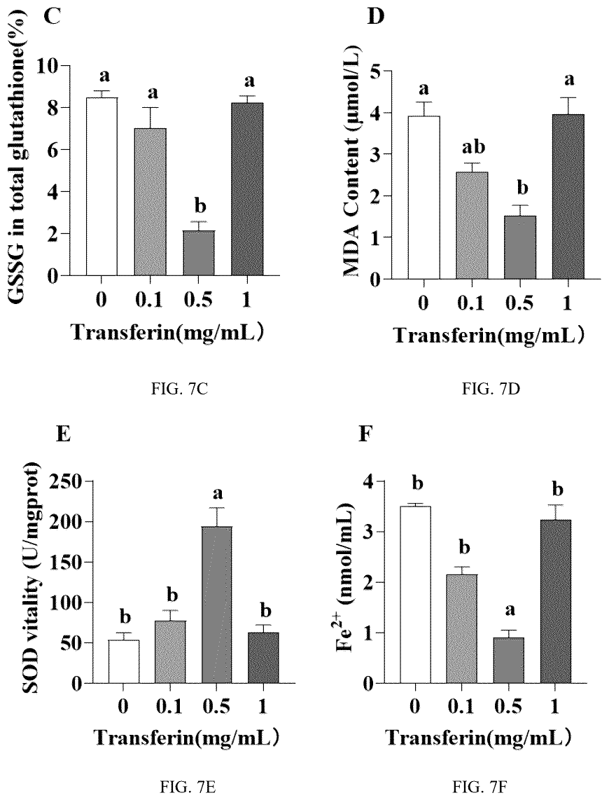

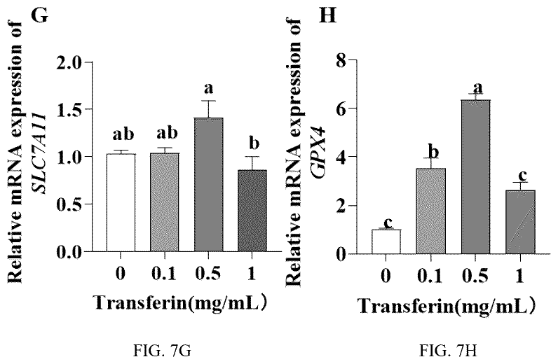

Results were shown in FIGS. 7A-7H: After PGCs are cultured for 3 d with ovotransferrin at different concentrations, there is no significant difference in terms of a total GSH content in PGCs among the ovotransferrin treatment groups, as shown in FIG. 7A (P>0.05). With the increase of a concentration of ovotransferrin, reduced GSH first increases and then decreases (FIG. 7B). With the increase of a concentration of ovotransferrin, oxidized GSSG first decreases and then increases (FIG. 7C). With the increase of a concentration of ovotransferrin, an MDA content first decreases and then increases (FIG. 7D). With the increase of a concentration of ovotransferrin, an activity of SOD first increases and then decreases (FIG. 7E). With the increase of a concentration of ovotransferrin, a ferrous ion content first decreases and then increases. The results show that ovotransferrin at an appropriate concentration can improve the oxidation resistance of PGCs and reduce the damage degree of PGCs, thereby affecting a ferrous ion content in PGCs (FIG. 7F). It is well known that GPX4 and SLC7A11 are genes for negative regulation of ferroptosis. As shown in FIG. 7G to FIG. 7H, ovotransferrin can inhibit the ferroptosis of PGCs. When a concentration of ovotransferrin is 0.5 mg/mL, there are the strongest oxidation resistance, the lowest MDA level, and the highest SOD level. Therefore, it can be seen that ovotransferrin can inhibit the ferroptosis of PGCs and improve the viability of PGCs.

The above are merely preferred examples of the present disclosure, and not intended to limit the present disclosure. Any modifications, equivalent replacements, and improvements made within the spirit and principle of the present disclosure should fall within the protection scope of the present disclosure.

What is claimed is:

1. A chicken serum-free medium for promoting proliferation of chicken primordial germ cells (PGCs), wherein in the chicken serum-free medium, ovotransferrin is used instead of chicken serum.

2. The chicken serum-free medium for promoting the proliferation of the chicken PGCs according to claim 1, wherein the chicken serum-free medium further comprises a Dulbecco's Modified Eagle Medium (DMEM), ultrafiltration water, and additives; and the additives comprise a concentrated calcium chloride stock solution, a B-27™ additive, a GlutaMax additive, an MEM non-essential amino acids solution, 2-mercaptoethanol, an EmbryoMax® nucleoside, sodium pyruvate, albumin, sodium heparin, basic fibroblast growth factor, activin A, and a penicillin-streptomycin mixed solution.

3. The chicken serum-free medium for promoting the proliferation of the chicken PGCs according to claim 1, wherein a concentration of the ovotransferrin in the chicken serum-free medium is 0.1 mg/mL to 1 mg/mL.

4. The chicken serum-free medium for promoting the proliferation of the chicken PGCs according to claim 3, wherein the concentration of the ovotransferrin in the chicken serum-free medium is 0.5 mg/mL.

5. A method for promoting proliferation of chicken PGCs, comprising using the chicken serum-free medium according to claim 1.

6. A method for promoting proliferation of chicken PGCs, comprising the following steps: inoculating the chicken PGCs into the chicken serum-free medium according to claim 1, and culturing at 37° C. for a predetermined period of time until a required number of the chicken PGCs are produced.

7. The method according to claim 5, wherein the chicken serum-free medium further comprises a Dulbecco's Modified Eagle Medium (DMEM), ultrafiltration water, and additives; and the additives comprise a concentrated calcium chloride stock solution, a B-27™ additive, a GlutaMax additive, an MEM non-essential amino acids solution, 2-mercaptoethanol, an EmbryoMax® nucleoside, sodium pyruvate, albumin, sodium heparin, basic fibroblast growth factor, activin A, and a penicillin-streptomycin mixed solution.

8. The method according to claim 5, wherein a concentration of the ovotransferrin in the chicken serum-free medium is 0.1 mg/mL to 1 mg/mL.

9. The method according to claim 8, wherein the concentration of the ovotransferrin in the chicken serum-free medium is 0.5 mg/mL.

10. The method according to claim 6, wherein the chicken serum-free medium further comprises a Dulbecco's Modified Eagle Medium (DMEM), ultrafiltration water, and additives; and the additives comprise a concentrated calcium chloride stock solution, a B-27™ additive, a GlutaMax additive, an MEM non-essential amino acids solution, 2-mercaptoethanol, an EmbryoMax® nucleoside, sodium pyruvate, albumin, sodium heparin, basic fibroblast growth factor, activin A, and a penicillin-streptomycin mixed solution.

11. The method according to claim 6, wherein a concentration of the ovotransferrin in the chicken serum-free medium is 0.1 mg/mL to 1 mg/mL.

12. The method according to claim 11, wherein the concentration of the ovotransferrin in the chicken serum-free medium is 0.5 mg/mL.
